(19) United States
(12) Patent Application Publication
(10) Pub. No.: US 2025/0388849 A1
(43) Pub. Date: Dec. 25, 2025

(54) INTEGRATED CULTURE-FREEZE DRYING METHOD FOR BIFIDOBACTERIUM LONGUM SUBSPECIES INFANTIS B8762

(71) Applicant: Inner Mongolia Agricultural University, Hothot (CN)

(72) Inventors: Heping Zhang, Hothot (CN); Guoqiang Yao, Hothot (CN); Kailong Liu, Hothot (CN); Feiyan Zhao, Hothot (CN)

(73) Assignee: Inner Mongolia Agricultural University, Hothot (CN)

(21) Appl. No.: 19/316,971

(22) Filed: Sep. 2, 2025

(51) Int. Cl. C12N 1/04; C12N 1/02; C12N 1/20; C12Q 1/06; C12R 1/01

(52) U.S. Cl. CPC C12N 1/04; C12N 1/02; C12N 1/20; C12Q 1/06; C12R 2001/01

(57) ABSTRACT

The present disclosure provides an integrated culture-freeze drying method for Bifidobacterium longum subsp. infantis B8762, which belongs to the field of biotechnology. The method includes: activating a strain in a carbon-source-free modified MRS culture medium and inoculating the activated strain into a fresh culture medium; adding one of D-lactose, D-sucrose or D-maltose; monitoring a growth curve, and building kinetic models in combination with metabolite analysis; collecting bacterial sludge after culture to a stationary phase, preparing a bacterial suspension, and testing a bacterial cell size, the number of growth generations, colony activity and a freeze-drying survival rate; and finally, performing vacuum freeze drying, and monitoring enzyme activity during storage. The method provided by the present disclosure can significantly improve the freeze-drying survival rate, delay attenuation of enzyme activity in a storage phase, and optimize growth and cell activity.

### TECHNICAL FIELD

The present disclosure relates to the field of biotechnology, and in particular to an integrated culture-freeze drying method for Bifidobacterium longum subsp. infantis B8762.

### BACKGROUND

Probiotics, as active microorganisms beneficial to host health, play a significant role in maintaining intestinal microecological balance, promoting immune function development and regulating nutritional metabolism. Among the probiotics, Bifidobacterium longum subsp. infantis has become a research hotspot in the field of biotechnology due to its special adaptability to infant intestinal health.

However, the strain faces key technical bottlenecks in the process of industrial production and storage: firstly, as an anaerobic and nutrient-demanding strain, cell membrane rupture and inactivation of key metabolic enzymes are prone to be caused during vacuum freeze drying due to low temperature stress, ice crystal formation and dehydration damage, which significantly reduces the survival rate of viable bacteria. Secondly, in existing culture systems, a conventional MRS culture medium does not optimize the correlation between carbon source type and freeze-drying resistance of bacterial cells. Thus, any improvement achieved by only adding freeze-drying protective agents in later phases is limited, which leads to excessive activity attenuation during storage of the strain, which seriously restricts industrial applications.

Therefore, it is of great significance to develop an integrated method which can enhance self-resistance of bacterial cells through precise regulation of a carbon source during culturing and combines with an optimized freeze-drying process, so as to improve the freeze-drying survival rate and storage stability of the Bifidobacterium longum subsp. infantis.

### SUMMARY

An objective of the present disclosure is to provide an integrated culture-freeze drying method for Bifidobacterium longum subsp. infantis B8762. The method can significantly improve the freeze-drying survival rate, delay attenuation of enzyme activity in a storage phase, and optimize growth and conversion efficiency.

In order to achieve the above objective, the present disclosure provides an integrated culture-freeze drying method for Bifidobacterium longum subsp. infantis B8762. The method includes the following steps:
step S1. activating a strain in a carbon-source-free modified MRS culture medium to obtain a seed solution, and inoculating the seed solution to a fresh carbon-source-free modified MRS culture medium;
step S2. adding a carbon source into the carbon-source-free modified MRS culture medium;
step S3. monitoring a growth curve by using an online living cell sensor, building kinetic models in combination with metabolite analysis, and regulating activity of bacterial cells; and
step S4. centrifugally collecting bacterial sludge after culture to a stationary phase, mixing the bacterial sludge with a freeze-drying protective agent to prepare a bacterial suspension, testing a bacterial cell size, the number of growth generations, colony activity and a freeze-dry survival rate, and performing storage at 4-25° C. to monitor enzyme activity.

Preferably, in the step S1, the number of activation generations is 2-3, a temperature is 36-38° C., time is 23-25 h, and pH is 6.18-6.22.

Preferably, in the step S1, culture conditions are as follows: a temperature is 37±0.2° C., culture is performed under anaerobic conditions for 24±0.5 h, pressure is maintained with nitrogen, a rotation speed is set to 80 r/min, and pH is maintained at 5.90±0.02 by automatically feeding ammonia water.

Preferably, in the step S2, the carbon source is one of D-lactose, D-sucrose or D-maltose, and a final concentration in the D-lactose, D-sucrose or D-maltose is 60 g/L.

Preferably, in the step S2, components of the carbon-source-free modified MRS culture medium are 10.0 g of peptone, 8.0 g of beef extract powder, 4.0 g of yeast extract powder, 1.0 mL of Tween-80, 2.0 g of dipotassium hydrogen phosphate, 5.0 g of sodium acetate, 2.0 g of triamine citrate, 0.05 g of manganese sulfate, 0.2 g of magnesium sulfate, 0.5 g of L-cysteine hydrochloride, and 1 L of distilled water, and pH is 6.18-6.22.

Preferably, in the step S3, the metabolite analysis includes the following steps:
determining the organic acid content through a liquid chromatography triple quadrupole mass spectrometer; and
determining the content of C, H, O and N through a FlashSmart elemental analyzer, and building the kinetic models of bacterial cell growth, product synthesis and substrate consumption on the basis of a total stoichiometry equation.

Preferably, in the step S3, when the carbon source is the D-lactose, the kinetic models are as follows:

$${X}_{1} ( t ) = \frac{31.5806 {e}^{0.9822 t}}{14.7024 + {X}_{0} ( {e}^{0.9822 t} - 1 )} ,$$

$${P}_{1} = 0.146 5 [ \frac{{e}^{0.9822 t}}{1 - 0.1461 ( 1 - {e}^{0.9822 t} )} - 1 ] + 2.1944 ln [ 1 - \frac{2.1480 ( 1 - {e}^{0.9822 t} )}{14.7024} ] ,$$

$$and$$

$${S}_{1} = 49.1550 - 1.8926 [ \frac{{e}^{0.9822 t}}{1 - 0.1461 ( 1 - {e}^{0.9822 t} )} - 1 ] - 0.1183 ln [ 1 - \frac{2.1480 ( 1 - {e}^{0.9822 t} )}{14.7024} ] .$$

When the carbon source is the D-sucrose, the kinetic models are as follows:

$${X}_{2} ( t ) = \frac{11.7393 {e}^{0.4539 t}}{6.6606 + 1.7625 ( {e}^{0.4539 t} - 1 )} ,$$

$${P}_{2} = - 0.864 \; 3 [ \frac{{e}^{0.4539 t}}{1 - 0.2646 ( 1 - {e}^{0.4539 t} )} - 1 ] + 2.6678 ln [ 1 - \frac{1.7625 ( 1 - {e}^{0.4539 t} )}{6.6606} ] ,$$

$$and$$

$${S}_{2} = 49.475 - 2.8503 [ \frac{{e}^{0.4539 t}}{1 - 0.2646 ( 1 - {e}^{0.4539 t} )} - 1 ] + 0.0895 ln [ 1 - \frac{1.7625 ( 1 - {e}^{0.4539 t} )}{6.6606} ] .$$

When the carbon source is the D-maltose, the kinetic models are as follows:

$${X}_{3} ( t ) = \frac{59.8687 {e}^{0.5655 t}}{19.6697 + 3.0437 ( {e}^{0.5655 t} - 1 )} ,$$

$${P}_{3} = - 0.748 \; 8 [ \frac{{e}^{0.5655 t}}{1 - 0.1547 ( 1 - {e}^{0.5655 t} )} - 1 ] + 5.7183 ln [ 1 - \frac{3.0437 ( 1 - {e}^{0.5655 t} )}{19.6697} ] ,$$

$$and$$

$${S}_{3} = 47.2201 - 1.5581 [ \frac{{e}^{0.5655 t}}{1 - 0.1547 ( 1 - {e}^{0.5655 t} )} - 1 ] - 2.2505 ln [ 1 - \frac{3.0437 ( 1 - {e}^{0.5655 t} )}{19.6697} ] .$$

In the formulas, X₀ represents an initial concentration, X₁(t), X₂(t) and X₃(t) represent growth of the bacterial cells, P₁, P₂ and P₃ represent a yield or concentration of a product, S₁, S₂ and S₃ represent consumption of a substrate, and t represents a time variable.

Preferably, in the step S4, the method for testing a bacterial cell size include the following steps:

staining bacterial flora by using gram stain, observing and recording the morphology size of the bacterial cells at different phases, processing the bacterial cell size by using Image View, and then, performing data sorting and data fitting.

Preferably, in the step S4, a calculation formula for testing the number of growth generations is:

$$Number of growth generations = {Log}_{2}^{100} ( \frac{{N}_{f}}{{N}_{0}} ) .$$

In the formula, N_{f} represents a viable count in the stationary phase, and N₀ represents an initial viable count of the bacterial cells inoculated into the fresh carbon-source-free modified MRS culture medium with 2% inoculation amount.

Preferably, the method for testing colony activity in the bacterial suspension includes: testing cell activity through flow cytometry, and distinguishing living cell, damaged cell and dead cell subsets by a propidium iodide and SYTO™9 double-staining method; and determining key enzyme activity through a kit.

Therefore, by using the integrated culture-freeze drying method for Bifidobacterium longum subsp. infantis B8762, the present disclosure has the beneficial effects as follows:

(1) Through precise regulation of the carbon source in a culture stage, the cell membrane integrity, sugar uptake ability and key metabolic enzyme activity of the bacterial cells are enhanced, laying a foundation for high-quality bacterial cells in a freeze drying process. In a freeze drying phase, relying on stress resistance formed in early phase culture, the freeze-drying survival rate is significantly improved, and attenuation of enzyme activity in a storage phase is delayed, so as to realize full-cycle retention of activity from culture to storage.

(2) Carbon source regulation prolongs the stationary phase of the bacterial cells, and improves a cell yield coefficient and substrate conversion efficiency. The carbon source type and the bacterial proliferation demand are accurately matched through growth kinetic models, and the biomass and the activity are both considered, such that the industrial production efficiency is improved.

### BRIEF DESCRIPTION OF THE DRAWINGS

FIG. 1 shows normal distribution trend change graphs of the morphology size of Bifidobacterium longum subsp. infantis B8762 in dynamic growth phases under different carbon source culture systems.

FIG. 2 shows the cell membrane integrity of bacterial cells before and after freeze drying in a sucrose culture group of the present disclosure, where A in FIG. 2 shows the cell membrane integrity before freeze drying in the sucrose culture group, and B in FIG. 2 shows the cell membrane integrity after freeze drying in the sucrose culture group.

FIG. 3 shows the cell membrane integrity of bacterial cells before and after freeze drying in a maltose culture group of the present disclosure, where A in FIG. 3 shows the cell membrane integrity of the maltose culture group before freeze drying, and B in FIG. 3 shows the cell membrane integrity of the maltose culture group after freeze drying.

FIG. 4 shows the cell membrane integrity of bacterial cells before and after freeze drying in a lactose culture group of the present disclosure, where A in FIG. 4 shows the cell membrane integrity of the lactose culture group before freeze drying, and B in FIG. 4 shows the cell membrane integrity of the lactose culture group after freeze drying.

FIG. 5 shows activity change histograms of fructose-6-phosphate phosphoketolase in different carbon source culture systems, where A in FIG. 5 is an activity change histogram of the fructose-6-phosphate phosphoketolase in different carbon source culture systems at a storage temperature of 4° C., and B in FIG. 5 is an activity change histogram of the fructose-6-phosphate phosphoketolase in different carbon source culture systems at a storage temperature of 25° C.

FIG. 6 is a growth curve graph of Bifidobacterium longum subsp. infantis B8762 under different carbon source systems.

FIG. 7 shows graphs of acid production characteristics of Bifidobacterium longum subsp. infantis B8762 under different culture carbon sources, where A in FIG. 7 shows the change of the lactic acid yield of three culture carbon sources with time, B in FIG. 7 shows the lactic acid yield of the three culture carbon sources at the fermentation endpoint, C in FIG. 7 shows the change of the acetic acid production yield of the three culture carbon sources with time, and D in FIG. 7 shows the acetic acid yield of the three culture carbon sources at the fermentation endpoint.

FIG. 8 shows graphs of the cell yield coefficient of Bifidobacterium longum subsp. infantis B8762 in different carbon source culture systems, where A in FIG. 8 shows the cell yield coefficient at different growth phases under different culture carbon sources, B in FIG. 8 shows the cell yield coefficient at a lag phase under different culture carbon sources, C in FIG. 8 shows the cell yield coefficient at a logarithmic phase under different culture carbon sources, D in FIG. 8 shows the cell yield coefficient at a stationary phase under different culture carbon sources, and E in FIG. 8 shows the cell yield coefficient at a death phase under different culture carbon sources.

FIG. 9 shows graphs of the product yield coefficient of Bifidobacterium longum subsp. infantis B8762 in different carbon source culture systems, where A in FIG. 9 shows the product yield coefficient at different growth phases under different culture carbon sources, B in FIG. 9 shows the cell product coefficient at a lag phase under different culture carbon sources, C in FIG. 9 shows the cell product coefficient at a logarithmic phase under different culture carbon sources, D in FIG. 9 shows the cell product coefficient at a stationary phase under different culture carbon sources, and E in FIG. 9 shows the cell product coefficient at a death phase under different culture carbon sources.

### DETAILED DESCRIPTIONS OF THE EMBODIMENTS

The technical solutions of the present disclosure are further described below with reference to the accompanying drawings and the examples.

An integrated culture-freeze drying method for Bifidobacterium longum subsp. infantis B8762 includes two main portions, namely culture and freeze drying.

### Example 1

A culture method for Bifidobacterium longum subsp. infantis B8762, including:

1. Strain activation and seed solution preparation

The strain of Bifidobacterium longum subsp. infantis B8762 at −80° C. was taken and activated for 2-3 generations (temperature of 36-38° C., time of 23-25 h, and pH of 6.18-6.22) in a carbon-source-free modified MRS culture medium to obtain a seed solution, and the seed solution was inoculated into a fresh carbon-source-free modified MRS culture medium according to a 5% inoculation amount and cultured for 24=0.5 h at 37=0.2° C. under an anaerobic condition (rotation speed of 80 r/min and maintaining pH at 5.90+0.02 with ammonia water). Components of the culture medium were 10.0 g of peptone, 8.0 g of beef extract powder, 4.0 g of yeast extract powder, 1.0 mL of Tween-80, 2.0 g of dipotassium hydrogen phosphate, 5.0 g of sodium acetate, 2.0 g of triamine citrate, 0.05 g of manganese sulfate, 0.2 g of magnesium sulfate, and 0.5 g of L-cysteine hydrochloride, distilled water was added to a constant volume of 1 L, and pH was adjusted to 6.20.

2. Carbon source regulation and bacterial cell culture

D-lactose, D-sucrose and D-maltose (final concentration being 60 g/L) was separately added in the culture medium, labeled as a D-lactose culture group, a D-sucrose culture group, and a D-maltose culture group respectively.

3. Metabolite analysis and kinetic model building

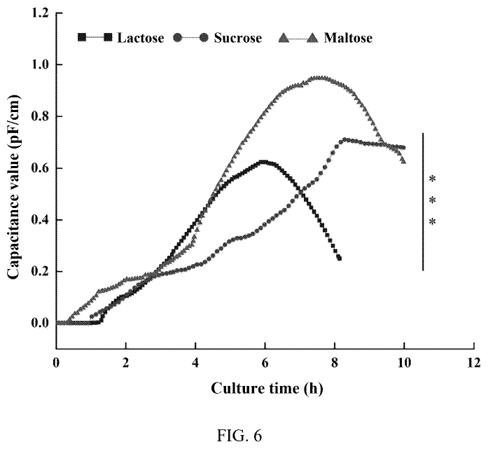

Growth curves (as shown in FIG. 6) were monitored by an online living cell sensor. Results show that the D-lactose culture group reaches the stationary phase at 5.73 h, and the living cell quantity is (0.623±0.005) pF/cm. After the stationary phase is maintained for 0.15 h, the death phase is started. The D-sucrose group reaches the stationary phase at 8.44 h, and the living cell quantity is (0.707±0.004) pF/cm. After the stationary phase is maintained for 1.22 h, the death phase is started. The D-maltose group reaches the stationary phase at 8.14 h, and the living cell quantity is (0.924±0.008) pF/cm. After the stationary phase is maintained for 0.53 h, the death phase is started. There is a significant difference among the three groups (P<0.05).

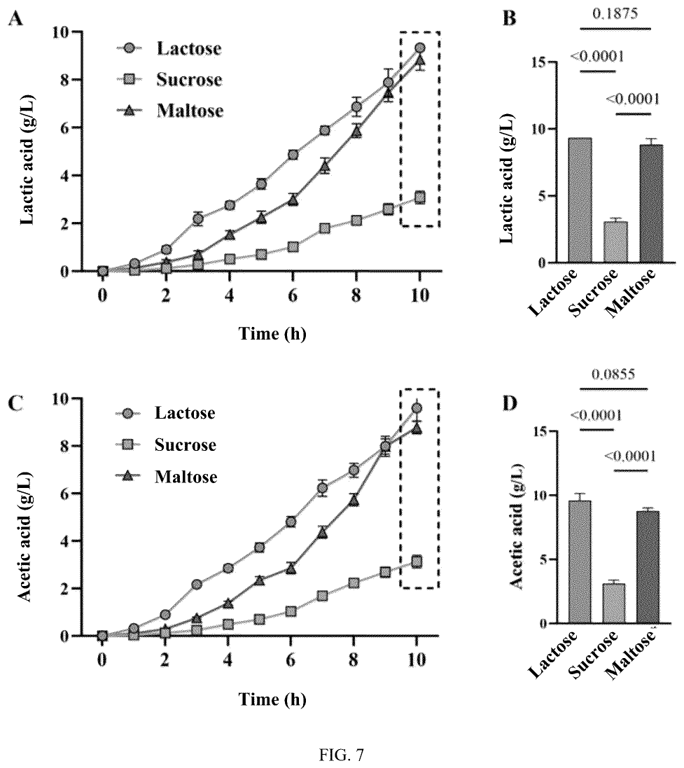

As shown in FIG. 7, metabolites are mainly lactic acid and acetic acid. The final yield of the D-sucrose culture group (lactic acid of 3.08±0.27 g/L and acetic acid of 3.13±0.18 g/L) is significantly low than that of the D-lactose culture group ((lactic acid of 9.33±0.27 g/L (P<0.0001) and acetic acid of 9.59±0.55 g/L (P<0.0001)) and the D-maltose culture group ((lactic acid of 8.83±0.22 g/L (P<0.0001) and acetic acid of 8.77±0.26 g/L (P<0.0001)). No significant difference is found in the final concentration of the lactic acid (P=0.1876) and the acetic acid (P=0.0855) between the D-lactose culture group and the D-maltose culture group.

Elements of C, H, O, and N of samples were analyzed by using a FlashSmart element analyzer. Considering the conservation of C, H, O, N and other elements, the activity of Bifidobacterium longum subsp. infantis B8762 under different culture carbon sources in a bioreactor can be expressed by the total stoichiometric equation:

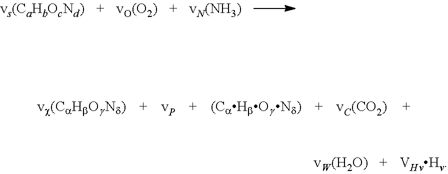

In the total stoichiometric equation, (C_{α}H_{b}O_{c}Nₐ) is a substrate general formula, (C_{α}H_{β}O_{γ}N_{δ}) is a cell general formula obtained based on elemental analysis, (C_{α}H_{β}O_{γ}N_{δ}) is a metabolite general formula, vₛ represents a stoichiometric coefficient of a substrate, v_{O} represents a stoichiometric coefficient of oxygen, v_{N} represents a stoichiometric coefficient of ammonia, v_{X} represents a stoichiometric coefficient of cell biomass, v_{P} represents a stoichiometric coefficient of a metabolite, v_{C} represents a stoichiometric coefficient of carbon dioxide, v_{W} represents a stoichiometric coefficient of water, V_{Hv} denotes a stoichiometric coefficient of hydrogen, α,α′and α represent the number of carbon atoms, β, β′, and b represent the number of hydrogen atoms, γ, γ′ and c represent the number of oxygen atoms, and δ, δ′ and d represent the number of nitrogen atoms.

The amount of unknown substances is calculated by the number of moles of known elements, and the calculation results are converted to mass or volume units. Then, by verifying whether the total mass and charge number of each element before and after the reaction are conserved, the calculation results are ensured to conform to the stoichiometric relationship, thus ensuring the accuracy and reliability of the whole calculation process.

Kinetic model building: Based on the total stoichiometry equation, the kinetic models of bacterial cell growth (X(t)), product synthesis (P) and substrate consumption(S) under three groups of carbon source systems were established respectively.

When the carbon source is the D-lactose, the kinetic models are as follows:

$${X}_{1} ( t ) = \frac{31.5806 {e}^{0.9822 t}}{14.7024 + {X}_{0} ( {e}^{0.9822 t} - 1 )} ,$$

$${P}_{1} = 0.146 5 [ \frac{{e}^{0.9822 t}}{1 - 0.1461 ( 1 - {e}^{0.9822 t} )} - 1 ] + 2.1944 ln [ 1 - \frac{2.1480 ( 1 - {e}^{0.9822 t} )}{14.7024} ] ,$$

$$and$$

$${S}_{1} = 49.1550 - 1.8926 [ \frac{{e}^{0.9822 t}}{1 - 0.1461 ( 1 - {e}^{0.9822 t} )} - 1 ] - 0.1183 ln [ 1 - \frac{2.1480 ( 1 - {e}^{0.9822 t} )}{14.7024} ] .$$

When the carbon source is the D-sucrose, the kinetic models are as follows:

$${X}_{2} ( t ) = \frac{11.7393 {e}^{0.4539 t}}{6.6606 + 1.7625 ( {e}^{0.4539 t} - 1 )} ,$$

$${P}_{2} = - 0.864 \; 3 [ \frac{{e}^{0.4539 t}}{1 - 0.2646 ( 1 - {e}^{0.4539 t} )} - 1 ] + 2.6678 ln [ 1 - \frac{1.7625 ( 1 - {e}^{0.4539 t} )}{6.6606} ] ,$$

$$and$$

$${S}_{2} = 49.475 - 2.8503 [ \frac{{e}^{0.4539 t}}{1 - 0.2646 ( 1 - {e}^{0.4539 t} )} - 1 ] + 0.0895 ln [ 1 - \frac{1.7625 ( 1 - {e}^{0.4539 t} )}{6.6606} ] .$$

When the carbon source is the D-maltose, the kinetic models are as follows:

$${X}_{3} ( t ) = \frac{59.8687 {e}^{0.5655 t}}{19.6697 + 3.0437 ( {e}^{0.5655 t} - 1 )} ,$$

$${P}_{3} = - 0.748 \; 8 [ \frac{{e}^{0.5655 t}}{1 - 0.1547 ( 1 - {e}^{0.5655 t} )} - 1 ] + 5.7183 ln [ 1 - \frac{3.0437 ( 1 - {e}^{0.5655 t} )}{19.6697} ] ,$$

$$and$$

$${S}_{3} = 47.2201 - 1.5581 [ \frac{{e}^{0.5655 t}}{1 - 0.1547 ( 1 - {e}^{0.5655 t} )} - 1 ] - 2.2505 ln [ 1 - \frac{3.0437 ( 1 - {e}^{0.5655 t} )}{19.6697} ] .$$

In the formulas, X₀ represents an initial concentration, X₁(t), X₂(t) and X₃(t) represent growth of the bacterial cells, P₁, P₂ and P₃ represent a yield or concentration of a product, S₁, S₂ and S₃ represent consumption of a substrate, and t represents a time variable.

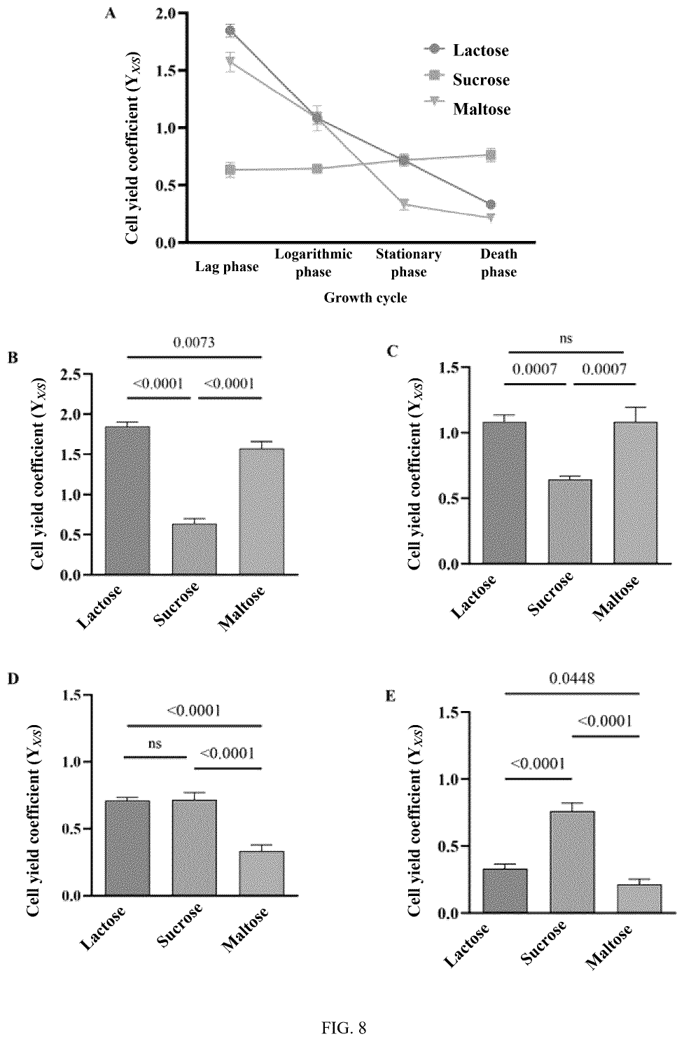

As shown in FIG. 8, the cell yield coefficient (Y_{X/S}) of the three carbon source culture groups is significantly different in the lag phase (AP), logarithmic phase (LP), stationary phase (SP) and death phase (DP):

lag phase: the lactose group (Y_{X/S}=1.85) and the maltose group (Y_{X/S}=1.54) are significantly higher than the sucrose group (0.63) (P<0.05);

logarithmic phase: Y_{X/S}=1.08 in the lactose group and the maltose group is significantly higher than that in the sucrose group (Y_{X/S}=0.64) (P=0.0007);

stationary phase: the sucrose group (Y_{X/S}-0.72) has no difference from the lactose group (Y_{X/S}=0.72), but is significantly higher than the maltose group (Y_{X/S}=0.37) (P<0.0001); and

death phase: the sucrose group (Y_{X/S}=0.76) is significantly higher than the lactose group (Y_{X/S}=0.33) and maltose group (Y_{X/S}=0.21) (P<0.0001).

The results show that the lactose group and the maltose group have higher cell yield coefficients in the lag phase and the logarithmic phase, and the sucrose group has better metabolic stability in the stationary phase and the death phase.

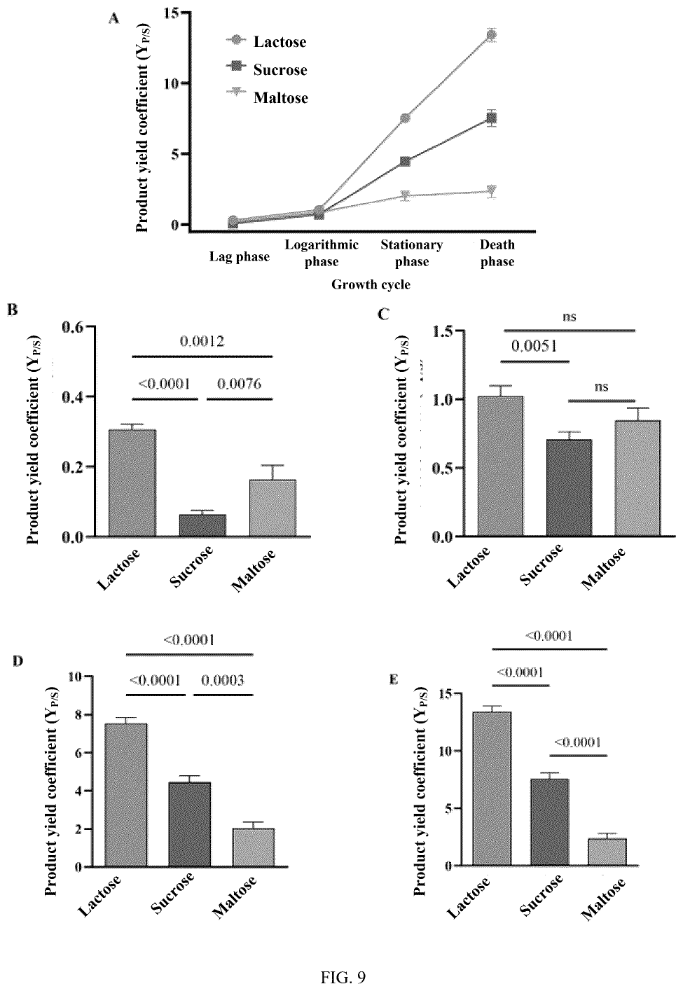

As shown in FIG. 9, the product yield coefficient (Y_{p/S}) of the three carbon source culture groups is significantly different at various growth phases:

lag phase (AP): the lactose group (Y_{p/S}=0.31) is significantly higher than the maltose group (Y_{p/S}=0.16) and the sucrose group (Y_{p/S}=0.06) (P<0.05);

logarithmic phase (LP): the lactose group (Y_{p/S}=1.02) is significantly higher than the sucrose group (Y_{p/S}=0.71) (P=0.0051), and the maltose group (Y_{p/S}=0.85) has no significant difference from other two groups; and

stationary phase (SP) and death phase (DP): the lactose group (Y_{p/S}=7.53 and Y_{p/S}=13.42 respectively) are significantly higher than the sucrose group (Y_{p/S}=4.50 and Y_{p/S}=7.53 respectively) and the maltose group (Y_{p/S}=2.03 and Y_{p/S}=2.36 respectively) (P<0.0001).

The results show that lactose as a carbon source has the highest product yield coefficient and the better product synthesis efficiency in the stationary phase and the death phase.

### Example 2

A freeze drying method for Bifidobacterium longum subsp. infantis B8762, including:

Preparation of bacterial suspension and index testing

(1) Preparation of bacterial suspension: a fermentation broth at the stationary phase was centrifuged at 4° C. and 4000 rpm for 15 min, supernatant was discarded, and bacterial sludge was washed with sterile PBS three times and mixed evenly with a freeze-drying protective agent according to a certain proportion to prepare the bacterial suspension.

(2) Index testing:

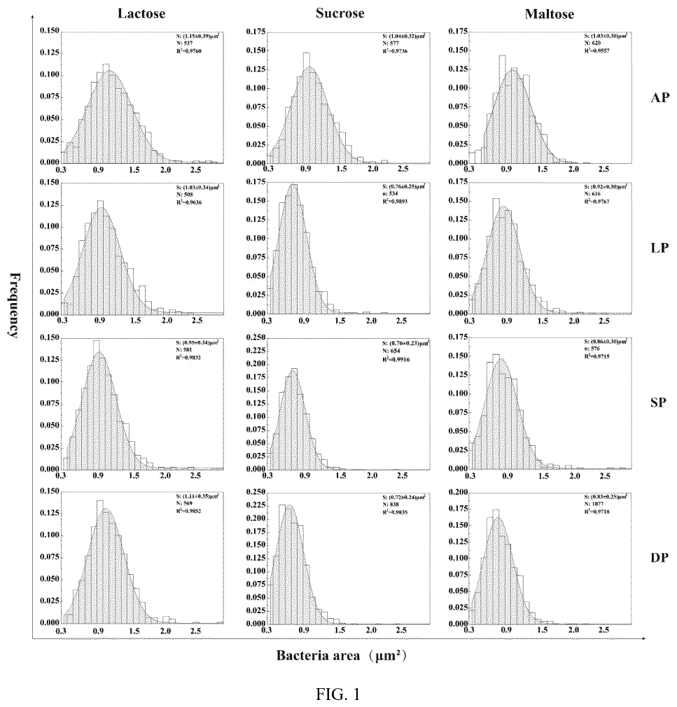

B8762 was sampled from the D-lactose culture group, the D-sucrose culture group and the D-maltose culture group at different growth phases. After Gram staining, a BX-53 microscope was used for marking the morphology size of the bacterial cells in different culture groups at different growth phases, and 300 fields were randomly selected from each phase. The size of the bacterial cells was processed by using ImageView. See FIG. 1 for the results of the normal distribution trend of the morphology size of the cells during cell growth.

The highest frequency areas are the lactose group (0.9-1.0) μm², the sucrose group (0.6-0.7) μm² and the maltose group (0.7-0.8) μm².

Lag phase: the average area (1.15±0.39 μm²) of the lactose group is significantly larger than those of the other two groups (P<0.05), showing Gaussian distribution (R²>0.94).

Logarithmic phase: the sucrose group has the largest decrease in size (26.9%, 0.76±0.25) μm², which is significantly smaller than those of the other two groups (P<0.001).

Death phase: the sizes of the three groups are (1.11±0.35), (0.72±0.24) and (0.83±0.25) μm² respectively, showing significant differences (P<0.05).

The results show that the carbon source affects the size of the bacterial cell size through regulation of the cell division cycle.

Number of growth generations: calculation was performed according to the formula:

$$Number of growth generations = {Log}_{2}^{100} ( \frac{{N}_{f}}{{N}_{0}} ) .$$

In the formula, N_{f} represents a viable count in the stationary phase, and N₀ represents an initial viable count of the bacterial cells inoculated into the fresh carbon-source-free modified MRS culture medium with 2% inoculation amount.

By quantifying the number of cell division generations of B8762 in different carbon source culture systems, the influence of carbon source metabolism characteristics on bacterial cell proliferation ability is revealed. Results are shown in Table 1.

TABLE 1
Number of bacterial cell division generations
of B8762 cultured with different carbon sources

| | | Viable count at | | |
|---|---|---|---|---|
| | Initial | beginning of | | |
| | viable count | stationary phase | | |
| | (×10⁸ | (×10⁹ | | Number of |
| Group | CFU/mL) | CFU/mL) | N_{f}/N₀ | generations |
| Lactose | 1.54 ± 0.17 | 4.40 ± 0.65 | 28.57 | 4.84 |
| culture group | | | | |
| Sucrose | | 7.28 ± 0.10 | 47.27 | 5.56 |
| culture group | | | | |
| Maltose | | 10.57 ± 0.10 | 68.64 | 6.10 |
| culture group | | | | |

As shown in Table 1, the numbers of division generations of the lactose culture group, the sucrose culture group and the maltose culture group are 4.84, 5.56 and 6.10 respectively, indicating that there are differences in cell proliferation ability under different carbon sources.

Vacuum freezing conditions are shown in Table 2.

TABLE 2
Vacuum freezing conditions

| | | | |
|---|---|---|---|
| Set | Set | Time | Vacuum |
| temperature | time | maintained | degree |
| (° C.) | (min) | (min) | (mbar) |

Pre-freezing stage

| | | | |
|---|---|---|---|
| −40 | 1-6 | 210-300 | / |

Primary drying

| | | | |
|---|---|---|---|
| −30 | 720-840 | 480-600 | 0.30 ± 0.02 |
| −25 | 360-480 | 300-480 | 0.25 ± 0.02 |
| −10 | 300-420 | 300-420 | 0.25 ± 0.02 |
| 10 | 180-240 | 120-180 | 0.25 ± 0.02 |

Desorption drying

| | | | |
|---|---|---|---|
| 20 | 1-180 | 1-120 | 0.25 ± 0.02 |
| 30 | 1-90 | 1-60 | 0.15 ± 0.02 |

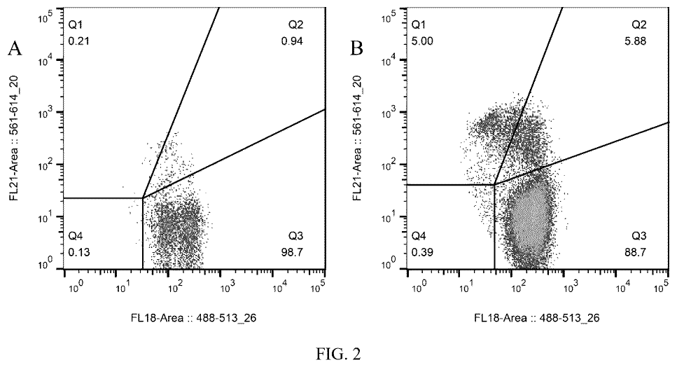

The change of the cell membrane integrity before and after freeze drying of B8762 is determined according to the chromatogram determined by using a flow cytometer, and results are shown in FIGS. 2-4.

As shown in FIG. 2 (sucrose culture group), undamaged cells account for 98.70% before freeze drying, which decreases to 88.70% (decrease of 10.00%) after freeze drying. The proportion of dead cells increases from 0.21% to 5.00% (increase of 4.79%).

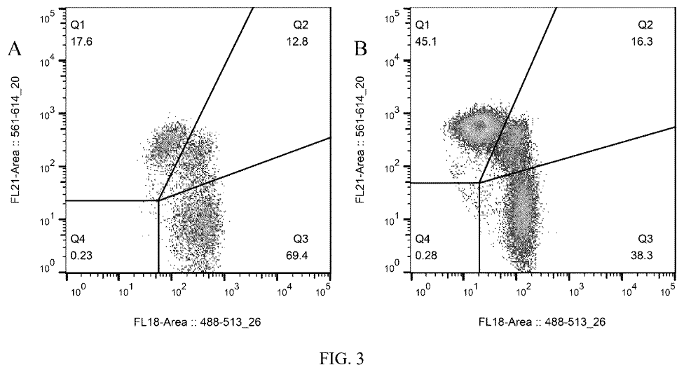

As shown in FIG. 3 (maltose culture group), undamaged cells account for 69.40% before freeze drying, which decreases to 38.30% (decrease of 31.10%) after freeze drying. The proportion of dead cells increases from 17.60% to 45.10% (increase of 27.50%).

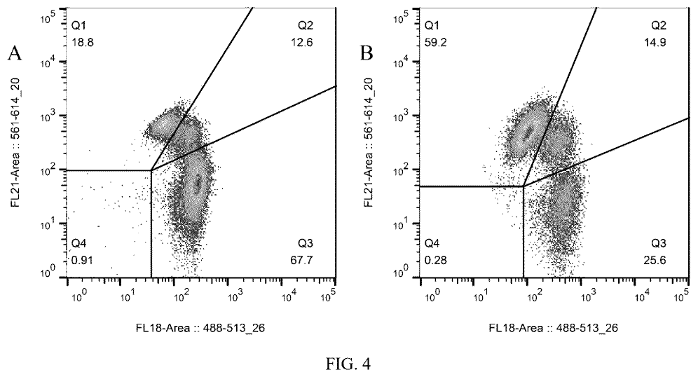

As shown in FIG. 4 (lactose culture group), undamaged cells account for 67.70% before freeze drying, which decreases to 25.60% (decrease of 42.10%) after freeze drying. The proportion of dead cells increases from 18.80% to 59.20% (increase of 40.40%).

Enzyme activity testing

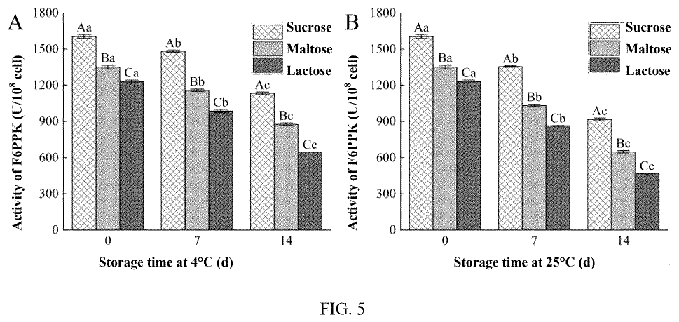

The activity of fructose-6-phosphate phosphoketolase (F6PPK) of bacterial cells of the Bifidobacterium longum subsp. infantis B8762 was measured by using a kit. Results are shown in FIG. 5.

As shown in FIG. 5, the activity of the F6PPK decreases with storage time, and the higher the temperature, the greater the decrease. At 4° C., the initial enzyme activity of the sucrose group is 1605 U/10⁸ cell, decreases to 1482 U/10⁸ cell after 7 days (decrease of 7.66%), and decreases to 1133 U/10⁸ cell after 14 days (decrease of 29.41%), which are lower than those of the maltose group (decrease of 35.11% after 14 days) and the lactose group (decrease of 47.40% after 14 days). At 25° C., the difference is more significant, the sucrose group decreases by 42.93% after 14 days, which is significantly lower than those of the maltose group (52.00%) and the lactose group (62.03%), indicating that bacterial powder in the sucrose group keeps better enzyme activity during storage, which is conductive to prolonging of a storage period and improvement of the quality.

Freeze-drying survival rate testing

Freeze-drying survival rate (%)=(weight of freeze-dried bacterial powder×viable count of bacterial powder)/(weight of pre-freezing bacterial solution×viable count of pre-freezing bacterial solution)×100%.

The freeze-drying survival rates of the lactose culture group, the sucrose culture group and the maltose culture group are shown in Table 3.

TABLE 3
Freeze-drying survival rates of B8762
cultured with different carbon sources

| | Freeze-drying | Yield (kg/t |
|---|---|---|
| Group | survival rate (%) | fermentation broth) |
| Lactose culture group | 50.45 | 6.09 |
| Sucrose culture group | 92.00 | 4.46 |
| Maltose culture group | 62.50 | 9.90 |

As can be seen from Table 3, when sucrose is used, the freeze-drying survival rate is 92.00%, the yield of single ton of fermentation broth is 4.46 kg. When the fermentation system is lactose, the freeze-drying survival rate is 50.45%, and the yield of single ton of fermentation broth is 6.09 kg. When the fermentation system is maltose, the freeze-drying survival rate is 62.50%, and the yield of single ton of fermentation broth is 9.90 kg.

According to the culture method provided by the present disclosure, a foundation is laid for the bacterial cells to accumulate stress resistant substances (such as enhancing cell membrane integrity and improving sugar uptake capacity) through precise regulation of carbon sources (D-lactose, D-sucrose or D-maltose) and optimization of the kinetic models. The freeze drying method significantly improves the freeze-drying survival rate by relying on the characteristics of the bacterial cells formed through preliminary culture and combining the freeze-drying protective agent and program optimization. Moreover, enzyme activity monitoring during storage shows that the integrated method can effectively delay the attenuation of key enzyme activity, realize the full cycle retention of the bacterial cell activity from culture to storage, and give consideration to industrial production efficiency and product stability.

What is claimed is:

1. An integrated culture-freeze drying method for Bifidobacterium longum subsp. infantis B8762, comprising that following steps:
step S1, activating a strain in a carbon-source-free modified MRS culture medium to obtain a seed solution, and inoculating the seed solution to a fresh carbon-source-free modified MRS culture medium;
step S2, adding a carbon source into the carbon-source-free modified MRS culture medium;
step S3, monitoring a growth curve by using an online living cell sensor, building kinetic models in combination with metabolite analysis, and regulating activity of bacterial cells; and
step S4, centrifugally collecting bacterial sludge after culture to a stationary phase, mixing the bacterial sludge with a freeze-drying protective agent to prepare a bacterial suspension, testing a bacterial cell size, the number of growth generations, colony activity and a freeze-dry survival rate, and performing storage at 4-25° C. to monitor enzyme activity.

2. The integrated culture-freeze drying method for Bifidobacterium longum subsp. infantis B8762 according to claim 1, wherein in step S1, the number of activation generations is 2-3, a temperature is 36-38° C., time is 23-25 h, and pH is 6.18-6.22.

3. The integrated culture-freeze drying method for Bifidobacterium longum subsp. infantis B8762 according to claim 1, wherein in the step S1, culture conditions are as follows: a temperature is 37±0.2° C., culture is performed under anaerobic conditions for 24±0.5 h, pressure is maintained with nitrogen, a rotation speed is set to 80 r/min, and pH is maintained at 5.90±0.02 by automatically feeding ammonia water.

4. The integrated culture-freeze drying method for Bifidobacterium longum subsp. infantis B8762 according to claim 1, wherein in step S2, the carbon source is one of D-lactose, D-sucrose or D-maltose, and a final concentration is 60 g/L.

5. The integrated culture-freeze drying method for Bifidobacterium longum subsp. infantis B8762 according to claim 1, wherein in step S2, components of the carbon-source-free modified MRS culture medium are 10.0 g of peptone, 8.0 g of beef extract powder, 4.0 g of yeast extract powder, 1.0 mL of Tween-80, 2.0 g of dipotassium hydrogen phosphate, 5.0 g of sodium acetate, 2.0 g of triamine citrate, 0.05 g of manganese sulfate, 0.2 g of magnesium sulfate, 0.5 g of L-cysteine hydrochloride, and 1 L of distilled water, and pH is 6.18-6.22.

6. The integrated culture-freeze drying method for Bifidobacterium longum subsp. infantis B8762 according to claim 1, wherein in step S3, the metabolite analysis comprises the following steps:
determining the organic acid content through a liquid chromatography triple quadrupole mass spectrometer; and
determining the content of C, H, O and N through an elemental analyzer, and building the kinetic models of bacterial cell growth, product synthesis and substrate consumption on the basis of a total stoichiometry equation.

7. The integrated culture-freeze drying method for Bifidobacterium longum subsp. infantis B8762 according to claim 6, wherein in the step S3, when the carbon source is the D-lactose, the kinetic models are as follows:
$${X}_{1} ( t ) = \frac{31.5806 {e}^{0.9822 t}}{14.7024 + {X}_{0} ( {e}^{0.9822 t} - 1 )} ,$$
$${P}_{1} = 0.146 5 [ \frac{{e}^{0.9822 t}}{1 - 0.1461 ( 1 - {e}^{0.9822 t} )} - 1 ] + 2.1944 ln [ 1 - \frac{2.1480 ( 1 - {e}^{0.9822 t} )}{14.7024} ] ,$$
$$and$$
$${S}_{1} = 49.1550 - 1.8926 [ \frac{{e}^{0.9822 t}}{1 - 0.1461 ( 1 - {e}^{0.9822 t} )} - 1 ] - 0.1183 ln [ 1 - \frac{2.1480 ( 1 - {e}^{0.9822 t} )}{14.7024} ] ;$$
when the carbon source is the D-sucrose, the kinetic models are as follows:
$${X}_{2} ( t ) = \frac{11.7393 {e}^{0.4539 t}}{6.6606 + 1.7625 ( {e}^{0.4539 t} - 1 )} ,$$
$${P}_{2} = - 0.864 \; 3 [ \frac{{e}^{0.4539 t}}{1 - 0.2646 ( 1 - {e}^{0.4539 t} )} - 1 ] + 2.6678 ln [ 1 - \frac{1.7625 ( 1 - {e}^{0.4539 t} )}{6.6606} ] ,$$
$$and$$
$${S}_{2} = 49.475 - 2.8503 [ \frac{{e}^{0.4539 t}}{1 - 0.2646 ( 1 - {e}^{0.4539 t} )} - 1 ] + 0.0895 ln [ 1 - \frac{1.7625 ( 1 - {e}^{0.4539 t} )}{6.6606} ] ;$$
and when the carbon source is the D-maltose, the kinetic models are as follows:
$${X}_{3} ( t ) = \frac{59.8687 {e}^{0.5655 t}}{19.6697 + 3.0437 ( {e}^{0.5655 t} - 1 )} ,$$
$${P}_{3} = - 0.748 \; 8 [ \frac{{e}^{0.5655 t}}{1 - 0.1547 ( 1 - {e}^{0.5655 t} )} - 1 ] + 5.7183 ln [ 1 - \frac{3.0437 ( 1 - {e}^{0.5655 t} )}{19.6697} ] ,$$
$$and$$
$${S}_{3} = 47.2201 - 1.5581 [ \frac{{e}^{0.5655 t}}{1 - 0.1547 ( 1 - {e}^{0.5655 t} )} - 1 ] - 2.2505 ln [ 1 - \frac{3.0437 ( 1 - {e}^{0.5655 t} )}{19.6697} ] ,$$
wherein, in the formulas, X₀ represents an initial concentration, X₁(t), X₂(t) and X₃(t) represent growth of the bacterial cells, P₁, P₂ and P₃ represent a yield or concentration of a product, S₁, S₂ and S₃ represent consumption of a substrate, and t represents a time variable.

8. The integrated culture-freeze drying method for Bifidobacterium longum subsp. infantis B8762 according to claim 1, wherein in step S4, the method for testing a bacterial cell size is as follows:
staining bacterial flora by using gram stain, observing and recording the morphology size of the bacterial cells at different phases, and processing the bacterial cell size by using image analysis software.

9. The integrated culture-freeze drying method for Bifidobacterium longum subsp. infantis B8762 according to claim 1, wherein in the step S4, a calculation formula for testing the number of growth generations is:
$$Number of growth generations = {Log}_{2}^{100} ( \frac{{N}_{f}}{{N}_{0}} ) ,$$
wherein, in the formula, N_{f} represents a viable count in the stationary phase, and N₀ represents an initial viable count of the bacterial cells inoculated into the fresh carbon-source-free modified MRS culture medium with 2% inoculation amount.

10. The integrated culture-freeze drying method for Bifidobacterium longum subsp. infantis B8762 according to claim 1, wherein the method for testing colony activity in the bacterial suspension comprises: testing cell activity through flow cytometry, and distinguishing living cell, damaged cell and dead cell subsets by a propidium iodide and SYTO™9 double-staining method; and
determining enzyme activity through a kit.
